(19) United States
(12) Patent Application Publication
(10) Pub. No.: US 2021/0128952 A1
(43) Pub. Date: May 6, 2021

(54) Sunscreen Application Assembly

(71) Applicant: Jaden Bober, Long Beach, CA (US)

(72) Inventor: Jaden Bober, Long Beach, CA (US)

(21) Appl. No.: 16/675,643

(22) Filed: Nov. 6, 2019

(51) Int. Cl. A61Q 17/04; A45D 44/00

(52) U.S. Cl. CPC A61Q 17/04; A45D 2200/1036; A45D 44/002

(57) ABSTRACT

A sunscreen application assembly for applying a fluid skin treatment to a user's face includes a mask that is positionable on a user's face. The mask includes a pair of eye cutouts, a nose flap and a mouth cutout. The mask is comprised of a deformable material thereby facilitating the mask to conform to the curvature of the user's face. A fluid skin treatment is applied to the mask thereby facilitating the fluid skin treatment to be applied to the user's face when the mask is positioned on the user's face. A protective sheet is bonded to the mask. The protective sheet is comprised of a fluid impermeable material to inhibit the fluid skin treatment from contacting the user's hands when the user applies the mask to the user's face.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to sunscreen devices and more particularly pertains to a new sunscreen device for applying a fluid skin treatment to a user's face.

### (2) Description of Related Art Including Information Disclosed Under 37 CFR 1.97 and 1.98

The prior art relates to sunscreen devices. Broadly speaking, the prior art covers various membranes that are positionable against human skin. Many of the membranes have cut outs for accommodating a user's eyes, nose and mouth. Thus, the membranes can be laid on the user's face without covering the user's eyes, nose and mouth. A fluid skin treatment, such as moisturizing lotion, cosmetic treatments or any other fluid skin treatment, is positioned in pools that are each located at various, strategic locations on the membrane. Thus, the pools of fluid skin treatment can be applied to corresponding strategic locations on the user's face without getting the fluid skin treatment onto the user's hands.

### BRIEF SUMMARY OF THE INVENTION

An embodiment of the disclosure meets the needs presented above by generally comprising a mask that is positionable on a user's face. The mask includes a pair of eye cutouts, a nose flap and a mouth cutout. The mask is comprised of a deformable material thereby facilitating the mask to conform to the curvature of the user's face. A fluid skin treatment is applied to the mask thereby facilitating the fluid skin treatment to be applied to the user's face when the mask is positioned on the user's face. A protective sheet is bonded to the mask. The protective sheet is comprised of a fluid impermeable material to inhibit the fluid skin treatment from contacting the user's hands when the user applies the mask to the user's face.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a front view of a sunscreen application assembly according to an embodiment of the disclosure.

FIG. 2 is a back view of an embodiment of the disclosure.

FIG. 3 is a cross sectional view taken along line 3-3 of FIG. 2 of an embodiment of the disclosure.

FIG. 4 is a perspective in-use view of an embodiment of the disclosure.

### DETAILED DESCRIPTION OF THE INVENTION

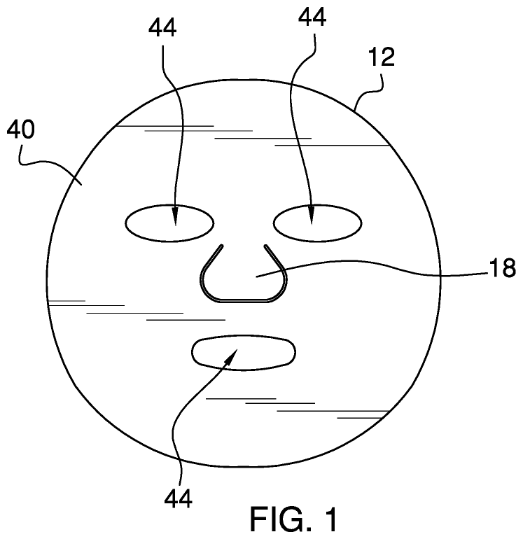

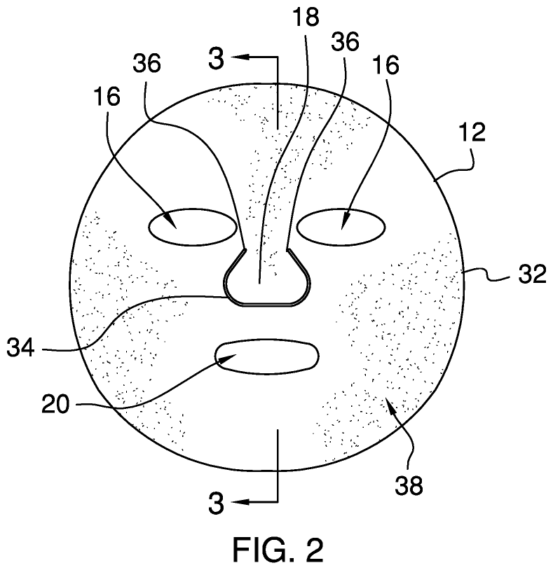

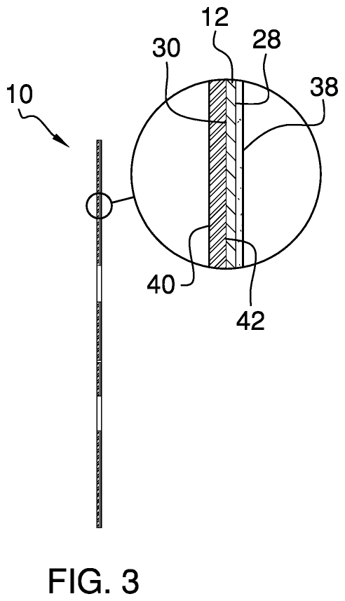

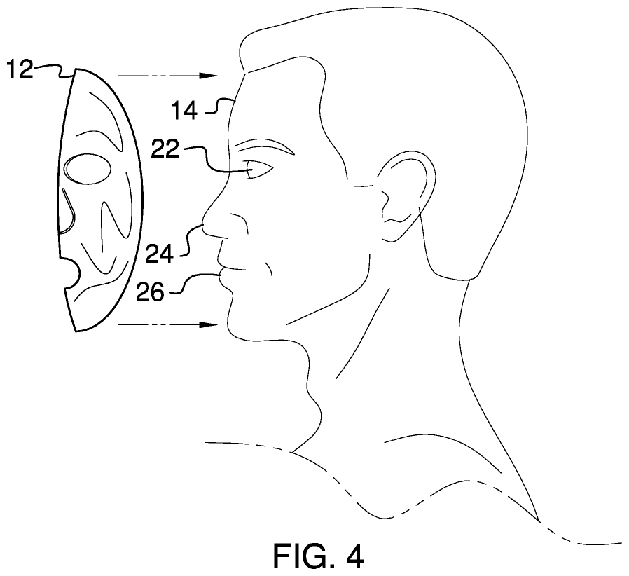

With reference now to the drawings, and in particular to FIGS. 1 through 4 thereof, a new sunscreen device embodying the principles and concepts of an embodiment of the disclosure and generally designated by the reference numeral 10 will be described.

As best illustrated in FIGS. 1 through 4, the sunscreen application assembly 10 generally comprises a mask 12 that is positionable on a user's face 14. The mask 12 includes a pair of eye cutouts 16, a nose flap 18 and a mouth cutout 20. Each of the eye cutouts 16 is aligned with a respective one of the user's eyes 22 when the mask 12 is positioned on the user's face 14. The nose flap 18 accommodates the user's nose 24 when the mask 12 is worn on the user's face 14. The mouth cutout 20 is aligned with the user's mouth 26 when the mask 12 is positioned on the user's face 14.

The mask 12 has a first surface 28, a second surface 30 and a perimeter edge 32 extending therebetween. The perimeter edge 32 is continuously arcuate about a center of the mask 12 such that the mask 12 has a circular shape. Each of the eye cutouts 16 extends through the first surface 28 and the second surface 30. The mask 12 has a cut 34 extending through the first surface 28 and the second surface 30 to define the nose flap 18. Moreover, each of a pair of terminal ends 36 of the cut 34 is positioned between the pair of eye cutouts 16. The cut 34 follows a curved line extending between the eye cutouts 16 and the mouth cutout 20 thereby facilitating the nose flap 18 to cover the user's nostrils 36. Additionally, the mask 12 is comprised of a deformable material, such as paper, thereby facilitating the mask 12 to conform to the curvature of the user's face 14.

A fluid skin treatment 38 is applied to the mask 12 thereby facilitating the fluid skin treatment 38 to be applied to the user's face 14 when the mask 12 is positioned on the user's face 14. The fluid skin treatment 38 completely covers the mask 12 thereby facilitating the fluid skin treatment 38 to be distributed over an entirety of the user's face 14. The fluid skin treatment 38 is positioned on the first surface 28 of the mask 12 and the fluid skin treatment 38 completely covers the first surface 28. The fluid skin treatment 38 may be sun screen, lotion or any other viscous material that is commonly applied to human skin.

A protective sheet 40 is bonded to the mask 12 and the protective sheet 40 is comprised of a fluid impermeable material such as foil or the like. The protective sheet 40 inhibits the fluid skin treatment 38 from contacting the user's hands when the user applies the mask 12 to the user's face 14. In this way the protective sheet 40 inhibits the user's hands from becoming slippery or greasy from the fluid skin treatment 38. The protective sheet 40 has a primary surface 42 and the primary surface 42 is bonded to the second surface 30 of the mask 12. The primary surface 42 completely covers the second surface 30 of the mask 12. Additionally, the protective sheet 40 has a plurality of cutouts 44 that is each aligned with a respective one of the nose flap 18, the eye cutouts 16 and the mouth cutout 20.

In use, the protective sheet 40 is gripped and the mask 12 is pressed against the user's face 14. In this way the fluid skin treatment 38 is applied to the user's face 14 without getting the fluid skin treatment 38 on the user's hands. Thus, the user's hands do not become slippery or greasy from the fluid skin treatment 38. The mask 12 is positioned on the user's face 14 such that the eye cutouts 16, the nose flap 18 and the mouth cutout 20 are aligned with the user's eyes 22, nose 24 and mouth 26. The mask 12 is removed from the user's face 14 after sufficient time has passed for the fluid skin treatment 38 to be transferred to the user's skin. The mask 12 and the protective sheet 40 are discarded once the mask 12 is removed from the user's face 14.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A sunscreen application assembly being configured to evenly apply sunscreen on a user's face without getting sunscreen on the user's hands, said assembly comprising:
a mask being positionable on a user's face, said mask including a pair of eye cutouts, a nose flap and a mouth cutout, each of said eye cutouts being aligned with a respective one of the user's eyes when said mask is positioned on the user's face, said nose flap accommodating the user's nose when said mask is worn on the user's face, said mouth cutout being aligned with the user's mouth when said mask is positioned on the user's face, said mask being comprised of a deformable material thereby facilitating said mask to conform to the curvature of the user's face;
a fluid skin treatment being applied to said mask thereby facilitating said fluid skin treatment to be applied to the user's face when said mask is positioned on the user's face, said fluid skin treatment completely covering said mask thereby facilitating said fluid skin treatment to be distributed over an entirety of the user's face; and
a protective sheet being bonded to said mask, said protective sheet being comprised of a fluid impermeable material to inhibit said fluid skin treatment from contacting the user's hands when the user applies said mask to the user's face wherein said protective sheet is configured to inhibit the user's hands from becoming slippery or greasy from said fluid skin treatment.

2. The assembly according to claim 1, wherein:
said mask has a first surface, a second surface and a perimeter edge extending therebetween, said perimeter edge being continuously arcuate about a center of said mask such that said mask has a circular shape, each of said eye cutouts extending through said first surface and said second surface; and
said mask has a cut extending through said first surface and said second surface to define said nose flap, each of a pair of terminal ends of said cut being positioned between said pair of eye cutouts having said cut following a curved line extending between said eye cutouts and said mouth cutout thereby facilitating said nose flap to cover the user's nostrils.

3. The assembly according to claim 2, wherein said fluid skin treatment is positioned on said first surface of said mask, said fluid skin treatment completely covering said first surface.

4. The assembly according to claim 2, wherein said protective sheet has a primary surface, said primary surface being bonded to said second surface of said mask, said primary surface completely covering said second surface.

5. A sunscreen application assembly being configured to evenly apply sunscreen on a user's face without getting sunscreen on the user's hands, said assembly comprising:
a mask being positionable on a user's face, said mask including a pair of eye cutouts, a nose flap and a mouth cutout, each of said eye cutouts being aligned with a respective one of the user's eyes when said mask is positioned on the user's face, said nose flap accommodating the user's nose when said mask is worn on the user's face, said mouth cutout being aligned with the user's mouth when said mask is positioned on the user's face, said mask having a first surface, a second surface and a perimeter edge extending therebetween, said perimeter edge being continuously arcuate about a center of said mask such that said mask has a circular shape, each of said eye cutouts extending through said first surface and said second surface, said mask having a cut extending through said first surface and said second surface to define said nose flap, each of a pair of terminal ends of said cut being positioned between said pair of eye cutouts having said cut following a curved line extending between said eye cutouts and said mouth cutout thereby facilitating said nose flap to cover the user's nostrils, said mask being comprised of a deformable material thereby facilitating said mask to conform to the curvature of the user's face;
a fluid skin treatment being applied to said mask thereby facilitating said fluid skin treatment to be applied to the user's face when said mask is positioned on the user's face, said fluid skin treatment completely covering said mask thereby facilitating said fluid skin treatment to be distributed over an entirety of the user's face, said fluid skin treatment being positioned on said first surface of said mask, said fluid skin treatment completely covering said first surface; and
a protective sheet being bonded to said mask, said protective sheet being comprised of a fluid impermeable material to inhibit said fluid skin treatment from contacting the user's hands when the user applies said mask to the user's face wherein said protective sheet is configured to inhibit the user's hands from becoming slippery or greasy from said fluid skin treatment, said protective sheet having a primary surface, said primary surface being bonded to said second surface of said mask, said primary surface completely covering said second surface, said protective sheet having a plurality of cutouts each being aligned with a respective one of said nose flap, said eye cutouts and said mouth cutout.

6. A method of applying a fluid skin treatment to a user's face without getting the fluid skin treatment on the user's hands, the steps of the method comprising:
providing a mask having a first surface, a second surfaced, a pair of eye cutouts, a nose flap and a mouth cutout;
providing a fluid skin treatment being positioned on said first surface of said mask having said fluid skin treatment completely covering said first surface, said fluid skin treatment completely covering said first surface;
providing a protective sheet being bonded to said mask;
gripping said protective sheet such that said protective sheet inhibits the fluid skin treatment from being applied to the user's hands;
pressing said mask against the user's face such that said fluid skin treatment is applied to the user's face;
removing said mask from the user's face leaving said fluid skin treatment behind; and
discarding said mask and said protective sheet.
